(19) United States
(12) Patent Application Publication
(10) Pub. No.: US 2026/0162797 A1
(43) Pub. Date: Jun. 11, 2026

(54) NETWORK MODEL TO PREDICT CANCER DRUG RESISTANCE CAUSED BY VARIANTS

(71) Applicant: George Mason University, Fairfax, VA (US)

(72) Inventor: Mohsin Saleet Jafri, Potomac, MD (US)

(21) Appl. No.: 19/413,325

(22) Filed: Dec. 9, 2025

Related U.S. Application Data
(60) Provisional application No. 63/730,235, filed on Dec. 10, 2024.

(51) Int. Cl. G16H 20/10; G16B 5/10; G16B 20/20; G16H 10/60

(52) U.S. Cl. CPC G16H 20/10; G16B 5/10; G16B 20/20; G16H 10/60

(57) ABSTRACT

The present disclosure generally relates to a system for prediction of drug resistance, caused by genetic variations of proteins, in cancer patients who are candidates for chemotherapy treatment. More specifically, the disclosure provides methods that combine proteins involved in cancer drug resistance into a set of signaling network models to predict cancer drug resistance based on single and multiple protein variants that can be found in a patient's tumor sample. Said method allows a physician to predict whether a patient is likely to respond to treatment with a given chemotherapeutic reagent.

### TECHNICAL FIELD

The present disclosure generally relates to a system for prediction of drug resistance, caused by genetic variations of proteins, in cancer patients who are candidates for chemotherapy treatment. More specifically, the disclosure provides methods that combine proteins involved in cancer drug resistance into a set of signaling network models to predict cancer drug resistance based on single and multiple protein variants that can be found in a patient's tumor sample. Said method allows a physician to predict whether a patient is likely to respond to treatment with a given chemotherapeutic reagent.

### BACKGROUND

Genetic variants resulting in cancer drug resistance account for >90% of cancer deaths. The proteins encoded by these genes participate in complex signaling networks. While some single variants have clinically actionable outcomes, there is limited guidance on how to understand the clinical significance of multiple variants. As a result, the first cancer drug therapy often fails endangering patient health through failed treatments, incurring unnecessary cost, and losing valuable time to contain the cancer. Accordingly, novel methods are needed to predict a patient's response to chemotherapy to assist the clinician's designing of optimal cancer treatment plans.

### SUMMARY

Disclosed herein is a system for predicting a cancer patient's response to chemotherapy that combines variant proteins involved in cancer drug resistance into a set of signaling network models to predict how the variant proteins will interact with one another for a given chemotherapy. In the provided method a graphical interface is generated to allow clinicians to easily understand the interactions and clinical recommendations to assist the oncologist in designing the optimal chemotherapeutic treatment plan.

According to an embodiment, the provided system comprises the individually performed steps of (i) variant prediction; (ii) creating a minimum viable product (MVP); and (iii) creation of a credential web platform for interacting with the developed technology. Said technology allows one to (i) predict drug resistance associated with a target protein having one or more a genetic variants either with known or predicted effect; (ii) predict drug resistance caused by genetic variations in proteins, that differ from the target protein, in an associated protein network; and (iii) develop a prototype with a well-defined user experience/interface The prototype allows the user to select the variants for the proteins relevant to their query as well as a drug of interest. The MVP prototype will return a prediction on the efficacy of the drug for the chosen set of variants.

The variant prediction step of the provided system applies machine learning to a feature set of phi and psi dihedral angles obtained from REST molecular dynamics simulations to predict functional changes associated with changes in the activity of variants. Said functional changes can be predicted with 90% accuracy or greater. Scientific literature data and predictions from this prediction step are used to formulate a Boolean network of a signaling network that is then used to predict the outcome of one or more variant profiles and their sensitivity to anticancer drugs.

As a second step, in the provided system for determining chemotherapeutic drug resistance in a cancer patient, a minimum viable product (MVP) is created to allow one (e.g. “user”) to enter (i) variant information on a patient's cancer and (ii) the drugs that are to be evaluated for efficacy for treatment of said cancer. To achieve this, the generated network model for a given pathway, as described above, is developed in FORTRAN, Python, or some other coding language to calculate the outcomes of the Boolean Model of the MVP. This network model is then connected to an HTML web page front end using a JavaScript or Flask framework. This model is then activated on an internal server. Such a web interface is designed to allow the user to select the desired specific gene variants for analysis and the one or more drugs to be tested. After the selections are made a submit button is clicked.

In an embodiment, the presently provided system is designed to address genetic variants associated with cancer that cause resistance to chemotherapy and added targeted therapy. In one embodiment, the disclosure provides a method for predicting the likelihood that cancer patients, who are candidates for chemotherapy, will respond to such treatment, comprising determining variant information associated with said cancer (tumor) and selecting one or more drugs that are to be evaluated for efficacy. Said variants and selected drug treatments are used to create an MVP that predicts the phenotype for multiple variants and the action of drugs.

In an embodiment a method is provided for determining a patient's prognosis for responding to a given chemotherapy comprising the steps of: (i) formulating a Boolean network of a given signaling network to be used to predict the outcomes of a variant profile and their sensitivity to one or more anticancer drugs; and (ii) creating a MVP that allows a user to enter variant information specific for the patient's cancer and drugs that are to be evaluated for efficacy.

### BRIEF DESCRIPTION OF THE FIGURES

FIG. 1. Signaling pathway diagram for BRAF and associated cellular growth and proliferation pathways involved in drug resistance. Arrows show activation and flat arrows (T-bar) show inhibition. Drug-resistant variants: Solid outline=excess activation and dashed outline=loss of inhibition. Ras represents a family of kinases including KRAS, NRAS and HRAS.

FIG. 2. Web page interface for MVP. Boxes are clicked as indicated by the checks and blue boxes.

FIG. 3. Web page interface for MVP. Results are returned after the “submit” button is clicked.

FIG. 4-B FIG. 4A. The genes associated with cell growth and proliferation are off. The inhibitory genes DUSP4 NF-1 and 4EBP1 are actively suppressing growth and proliferation. FIG. 4B. In control cell growth and cell cycle progression are turned off.

FIG. 5A-B. FIG. 5A The genes associated with cell growth and proliferation are on when EGF is present. FIG. 5B. In control cell growth and cell cycle survival are turned on.

FIG. 6A-B. FIG. 6A. The genes associated with cell growth and proliferation are turned off with the addition of the EGFR blocker cobimetinib. Even when EGF is present. FIG. 6B. In control cell growth and cell cycle survival are turned off with cobimetinib.

### DETAILED DESCRIPTION

Unless defined otherwise, all technical and scientific terms used herein have the same meaning as commonly understood to one of ordinary skill in the art to which this disclosure belongs. Although any methods, devices, and materials similar or equivalent to those described herein can be used in the practice or testing of the methods, devices and materials, the preferred methods, devices, and materials are now described.

### Definitions

The term “biological sample,” as used herein, refers to a sample obtained from an organism or from components (e.g., cells) of an organism. The sample may be of any biological tissue or fluid. The sample may be a “clinical sample” which is a sample derived from a patient.

As used herein, the term “biomarker” refers to a molecule that is associated either quantitatively or qualitatively with a biological change. Examples of biomarkers include polypeptides, proteins or fragments of a polypeptide or protein; and polynucleotides, such as a gene product, RNA or RNA fragment; and other body metabolites. In certain embodiments, a “biomarker” means a variant molecule that is present in a biological sample from a subject having a first phenotype (e.g., responding to drug treatment) as compared to a biological sample from a subject or group of subjects having a second phenotype (e.g., not responding to a drug treatment).

As used herein, the terms “indicates” or “correlates” (or “indicating” or “correlating,” or “indication” or “correlation,” depending on the context) in reference to a parameter, e.g., the level of expression of a biomarker gene in a sample from a may mean that the patient is likely, or unlikely, to respond to chemotherapy. In specific embodiments, the parameter may comprise the level of expression of one or more biomarkers as disclosed herein.

The terms “measuring” and “determining” are used interchangeably throughout and refer to methods which include obtaining or providing a patient sample and/or detecting the level of biomarker expression in a sample. In certain embodiments, the terms are also used interchangeably with the term “quantitating.”

The term “variant” or “variant protein” refers to a protein having a mutation that distinguishes from a wild-type version of the protein. Variant or variant protein may be used herein interchangeably with “mutant” or “mutant protein.” Variant genes refer to those nucleic acid molecules encoding for said variant proteins.

The present disclosure generally relates to profiling of tissue samples obtained from cancer patients who are candidates for chemotherapy treatment to determine the presence of variant proteins in the tissue sample. Said samples are obtained from patients and the presence of variant protein is determined. More specifically, the present disclosure provides methods, based on characterization of gene variants within a patient, which allows a physician to predict whether a patient is likely to respond well to treatment with a chemotherapeutic reagent.

The present disclosure provides a system of preparing a prognostic profile for a cancer patient using each of the disclosed steps, i.e., variant prediction and MVP creation, described below for predicting the response of a cancer patient to a given chemotherapeutic reagent. Additionally, each of the disclosed methods above may further comprise the step of creating a report summarizing the data obtained by said analysis. In yet another embodiment, the disclosed methods above may further comprise the administration of the chemotherapeutic reagent where it is determined that the patient is likely to respond to such drug treatment.

More specifically, disclosed herein is a system for predicting a cancer patient's response to chemotherapy that combines variant proteins involved in cancer drug resistance into a set of signaling network models to predict how the variant proteins will interact with one another for a given chemotherapy. In the provided method a graphical interface is generated to allow clinicians to easily understand the interactions and clinical recommendations to assist the oncologist in designing the optimal chemotherapeutic treatment plan.

According to an embodiment, the provided system comprises the individually performed steps of (i) variant prediction; (ii) creating a minimum viable product (MVP); and (iii) creation of a credential web platform for interacting with the developed technology. Said technology allows one to (i) predict drug resistance associated with a target protein having a genetic variation; (ii) predict drug resistance caused by genetic variations in proteins, that differ from the target protein, in an associated protein network; and (iii) develop a prototype with a well-defined user experience/interface

The variant prediction step of the provided system applies machine learning to a feature set of phi and psi dihedral angles obtained from REST molecular dynamics simulations to predict functional changes associated with changes in the activity of variants. Said functional changes can be predicted with 90% accuracy or greater. The data and predictions from this variant prediction step are used to formulate a Boolean network of a signaling network that is then used to predict the outcome of one or more variant profiles and their sensitivity to anticancer drugs.

In embodiments of the invention, said variant proteins can be those variants know to be directly associated with a given cancer. Such variant proteins include, for example, oncogenes, those variants found in in tumor suppressor genes and variants involved in DNA repair. Such gene variants include, but are not limited to, BRAF, KRAS, PTEN, APC, MEK-1, BMPR1A, BRCA1, BRCA2, MEN1, MLH1, MSH2, MSH6, MUTYH, NF2, PMS2, PTEN, RB1, RET, SDHB, SDHC, SDHD, SMAD4, STK11, TP53, TSC1, TSC2, VHL, and WT1. Variant proteins to be included in the signaling network may also include those proteins know to be associated with a cancer associated protein, or those variant proteins found to be part of a signaling pathway which contains the cancer associated protein.

The determination of variant protein information associated with a cancer patient can be determined by methods known in the art. In particular embodiments, the methods disclosed herein include collecting a biological sample, such as a primary colorectal tumor sample in which expression of a biomarker gene can be detected. Biological samples may be obtained from a subject by a variety of techniques including, for example, by scraping or swabbing an area, by using a needle to aspirate cells, or by removing a tissue sample (i.e., biopsy). Methods for collecting such biological samples are well known in the art. In some embodiments, a colorectal tumor sample is obtained by, for example, fine needle aspiration biopsy, core needle biopsy, or excisional biopsy. Fixative and staining solutions may be applied to the cells or tissues for preserving the specimen and for facilitating examination. Biological samples, particularly colorectal tumor samples, may be transferred to a glass slide for viewing under magnification. In one embodiment, the biological sample is a formalin-fixed, paraffin-embedded tissue sample, particularly a primary colorectal tumor sample.

The presence of the one or more variant proteins in a tissue sample derived from a cancer patient can be determined by methods known in the art. Methods for detecting expression of the variant genes/proteins disclosed herein include methods based on hybridization analysis of polynucleotides, methods based on sequencing of polynucleotides, immunohistochemistry methods, and proteomics-based methods. The methods generally detect expression products (e.g., mRNA or protein) of the variant gene or gene product. In preferred embodiments, PCR-based methods, such as reverse transcription PCR (RT-PCR), and array-based methods are used.

Anticancer drugs which can be tested for sensitivity include a wide range of drugs that work in different ways to stop cancer cells from growing and dividing. In non-limiting embodiments such drugs include those of several major classes, including alkylating agents, antimetabolites, anthracyclines, topoisomerase inhibitors, and mitotic inhibitors. Anticancer drugs include, but are not limited to BRAF inhibitors such as vemurafenib, dabrafenib, EGFR inhibitors such as cetuximab, MEK inhibitors such as cobimetinib and trametinib, PI3K inhibitors such as alpelisib, KRAS inhibitors such as sorafenib, Akt inhibitors such as BAY 1125976 and PI3K inhibitors such as AZD6482.

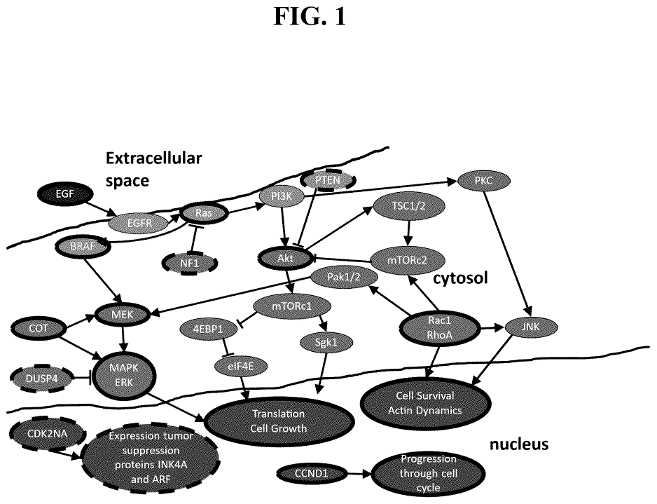

In a non-limiting embodiment, such a variant prediction step was used to accurately predict BRAF sensitivity or resistance to dabrafenib or vemurafenib. BRAF refers to a gene that codes for a protein involved in cell growth and division. (See, Xie et al., Combined Molecular Dynamics and Machine Learning to Predict Drug Resistance Causing Variants of BRAF in Colorectal Cancer, Molecules, 2025, 30 (17) 3556 which is incorporated herein in its entirety). When the BRAF gene is mutated, it can lead to uncontrolled cell growth and is associated with many types of cancer, including melanoma, lung, and thyroid cancers. The most common mutation is the V600E mutation, which can affect how the protein functions and makes it a target for certain cancer therapies. Accordingly, the present disclosure provides a method for detecting resistance to dabrafenib or vemurafenib in a cancer patient wherein in said patient is identified by detection of BRAF gene mutation. Further the variant prediction step was used to determine whether PTEN variants were normal or had a loss of function. PTEN is a gene that acts as a tumor suppressor by producing a protein that regulates cell growth. Mutations in the PTEN gene can lead to uncontrolled cell growth, increasing the risk of various cancers. As described below, in a specific aspect, it was demonstrated that the loss of function of PTEN removed inhibition of AKT which can lead to uncontrolled tumor growth in spite of blocking upstream proteins such as EGFR or BRAF (FIG. 1). Accordingly, the present disclosure provides a method for detecting resistance to vemurafenib, dabrafenib, cobimetinib, and cetuximabin a cancer patient wherein in said patient is identified by detection of a PTEN gene mutation. Further, prognosis for KRAS variants was successfully made through the variant prediction step. KRAS refers to a gene that produces a protein involved in cell growth, division, and death. Mutations in this gene can cause it to become overactive, leading to uncontrolled cell growth and the development of various cancers, including lung, colorectal, and pancreatic cancers. The prognosis of KRAS variants was shown to correlated with KRAS gain of function variants and responsiveness to EGFR inhibitors that work to turn off KRAS. Accordingly, the present disclosure provides a method for detecting resistance to vemurafenib, dabrafenib, cobimetinib, and cetuximab in a cancer patient wherein in said patient is identified by detection of a KRAS gene mutation.

In the provided system for determining chemotherapeutic drug resistance associated with the presence of one or more variant proteins in a cancer patient, a minimum viable product (MVP) is created to allow one (e.g. “user”) to enter (i) single or multiple variant information on a patients cancer and (ii) the drugs that are to be evaluated for efficacy for treatment of said cancer. To achieve this, the generated network model for a given pathway, as described above, is developed in FORTRAN or Python code to perform Boolean model calculations of the MVP. This network model is then connected to an HTML web page front end using a JavaScript or Flask framework. This model is then activated on an internal server. Such a web interface is designed to allow the user to select the desired specific gene variants for analysis and the one or more drugs to be tested. After the selections are made a submit button is clicked.

The present disclosure provides a method of preparing a prognostic profile for a cancer patient, comprising the steps of: (i) formulating a Boolean network of a given signaling network to be used to predict the outcomes of a variant profile and their sensitivity to one or more anticancer drugs; and (ii) creating a MVP that allows a user to enter variant information specific for the patient's cancer and drugs that are to be evaluated for efficacy; and (iii) creating a report summarizing the data obtained by said prognostic profiling.

Unless otherwise defined, all technical and scientific terms used herein have the same meaning as commonly understood by one skilled in the art. Although methods and materials similar to or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLE

Signaling pathway diagram for BRAF and associated pathways involved in drug resistance is shown in FIG. 1. Arrows show activation and flat arrows (T-bar) show inhibition. Drug-resistant variants: Solid outline=excess activation and dashed outline=loss of inhibition. Ras represents a family of kinases including KRAS, NRAS and HRAS.

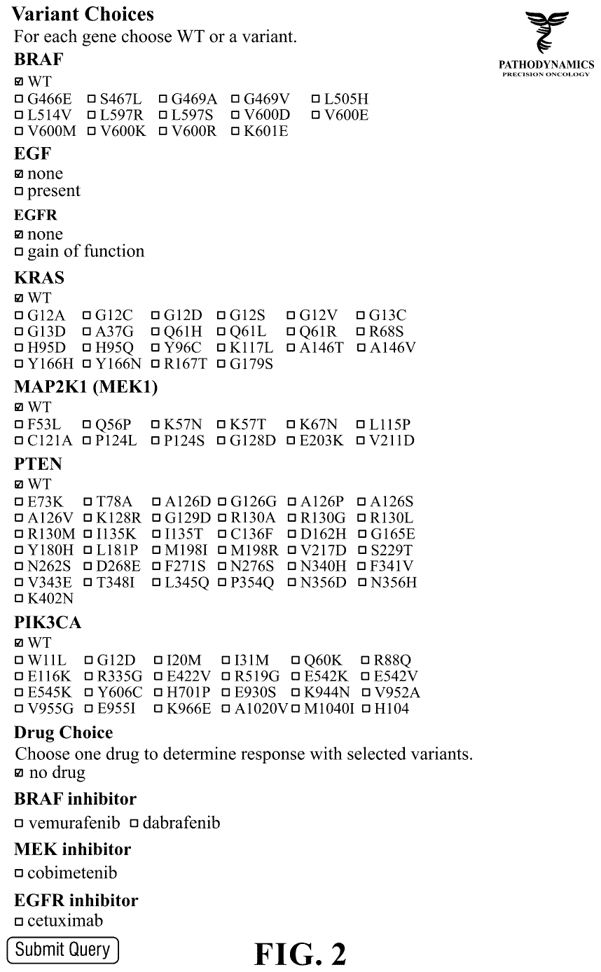

FIG. 2. Depicts web page interface for MVP. Boxes are clicked as indicated by the checks and blue boxes. Results are returned after the “submit” button is clicked. The MVP allows the user to enter variant information on a patient's cancer and the drugs that are to be evaluated for efficacy. To this end, the network model for the signaling pathway shown in FIG. 1 was first developed in FORTRAN and then was converted to Python code to serve as the back end of the MVP. This was connected to an HTML web page front end using the Flask framework. This was activated on an internal server. The web interface was designed to allow the user to select the gene variants desired and the drug that is to be tested. After the boxes are checked the submit button is clicked. The HTML web page graphical user interface (GUI) inputs are read by the Python back end using the Flask microweb framework that allows communication between the GUI front end and the back end. FIG. 2 shows the web page where the user will input the variant and drug information by clicking the boxes. The signaling pathway diagram may be implemented by a signaling network model, which may be implemented in various embodiments by a Boolean network model. A Boolean network model may be implemented in various ways using data structures and using computer-executable instructions. The data structures may represent genes or proteins, among other things, and the binary variables in the data structures may have values indicating whether the gene or proteins, among other things, activated or inhibited. In various embodiments, the data structures may include signaling connections to other data structures, which represent the connections in the signaling pathway diagram. The signaling connections may also be represented in the Boolean network model and by binary variables. Such and other implementations and variations are contemplated to be within the scope of the present disclosure.

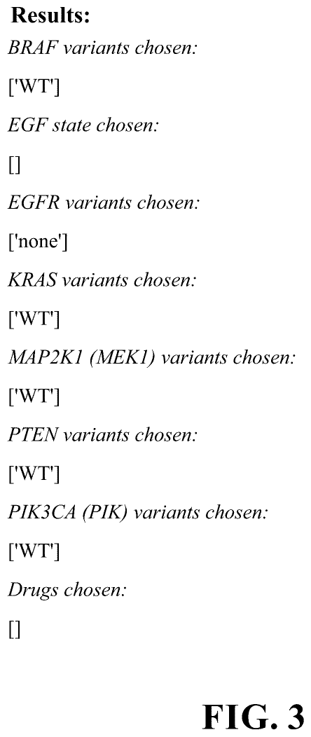

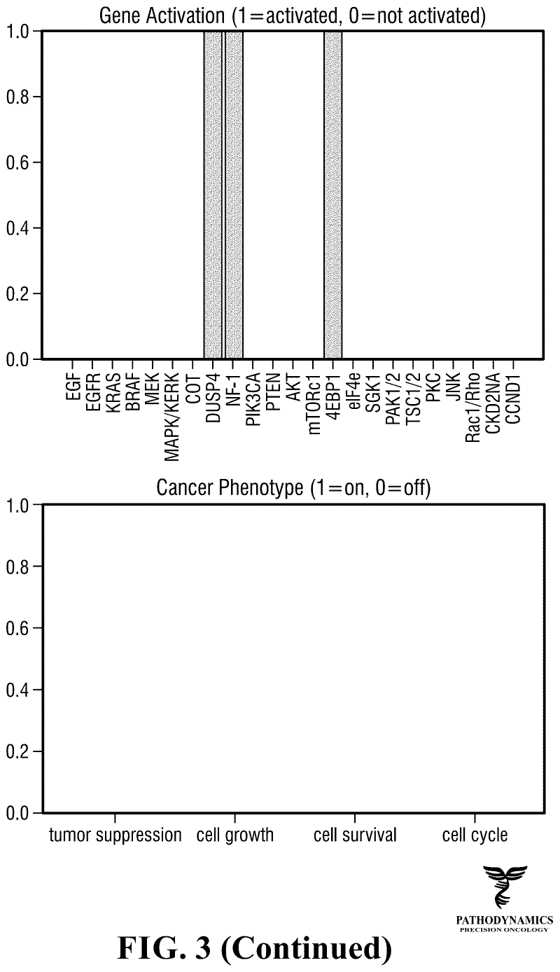

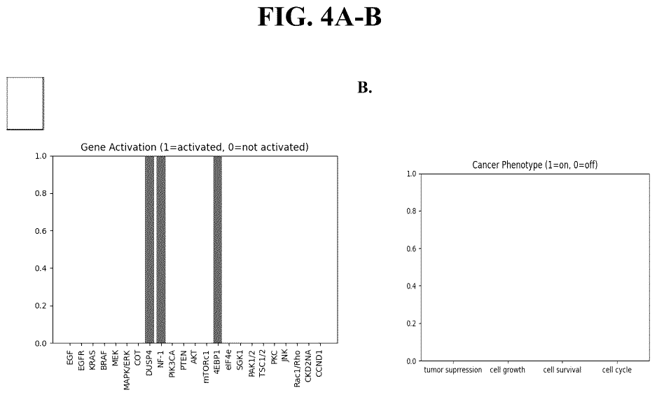

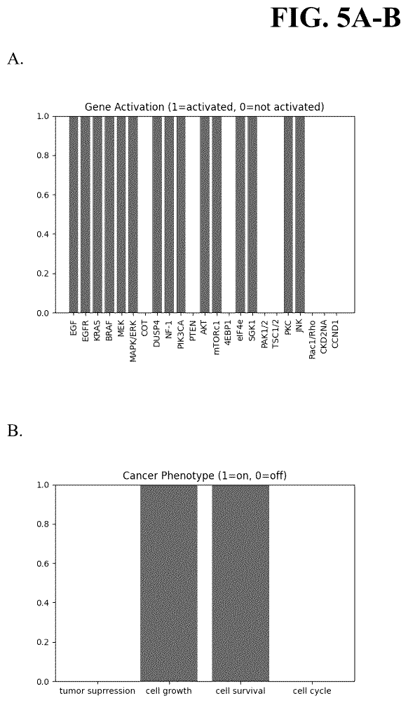

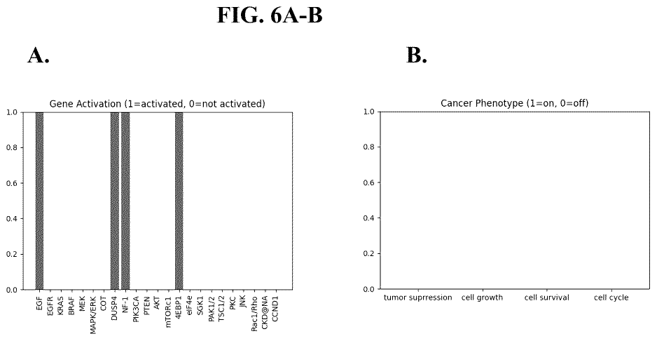

The inputs chosen, the genes activated, and the phenotypic response are returned almost instantaneously to the same window (FIG. 3). FIG. 4 shows the MVP output for a normal cell. The growth pathways are not activated (FIG. 4A) and the phenotype does not indicate growth (FIG. 4B). When the growth factor EGF is activated, it binds to activate the EGFR (EGF receptor), and this leads to the activation of pathways leading to growth and cell survival (FIG. 5A-B). When the EFFR inhibitor cobimetinib is added, growth and cell survival pathways are turned off even while EGF remains in the system (FIG. 5A-B).

Table 1 and Table 2 summarize some of the possible inputs to the model (shown in FIG. 1). In the normal unstimulated cell, the growth and proliferation pathways are turned off (not activated). When EGF, is added it activates the pathway EGFR→KRAS→BRAF and PI3K, and the signaling cascade occurs that results in growth and cell survival (FIG. 1). The response to EGF can be blocked by the EGFR inhibitor cobimetinib. The V600E BRAF mutation accounts for 8-10% of metastatic colorectal cancer patients. When the V600E variant is introduced, cell growth occurs without activating KRAS. The effects of this phenotype can be blocked by the BRAF inhibitor vemurafenib. However, with the V601E BRAF variant, the BRAF inhibitor drugs vemurafenib and dabrafenib do not work and still give the cancer phenotype of cell growth similar to the undrugged BRAF V600E variant result in Table 1 and Table 2. The MVP can also consider other variants such as the KRAS Q61H variant that leads to cell growth and survival which activates growth through pathways involving the pathway KRAS→BRAF and KRAS→PI3K (FIG. 1). The MVP also predicts the phenotype for multiple variants and the action of drugs. When the BRAF V600E and KRAS Q61H variants are present and the BRAF inhibitor vemurafenib is administered, the growth and cell survival pathways are still activated by the activated KRAS through PI3K, even though the BRAF→MEK→MAPK/ERK activation pathway is inhibited.

### EXAMPLE 2

The signaling network involved in gastrointestinal cancer is shown in FIG. 1. In certain cancers BRAF inhibitors are used to address gain-of-function variants in the proto-oncogene BRAF. However, a second mutation in MEK can cause the BRAF inhibitor to fail. For this reason, in colorectal cancer both BRAF inhibitors and MEK inhibitors have been shown to offer better patient outcomes. A tool such as this will indicate for which patients such a combination therapy is needed. To facilitate the clinical interpretation of the interaction of the different variants found in a patient's cancer molecular diagnostic test a network model that gives a quantitative output such as growth and proliferation is developed. This will include the curated variants whose effect on drug resistance is known as well as the predicted variants as disclosed above. The effect on growth and proliferation of considering only the curated vs the curated+predicted variants can be presented as well as possible drug interventions including combination therapies.

In addition to these proteins, there are 24 genes in the American College of Medical Genetics and Genomics (ACMG v2.0) list of germline cancer associated genes (APC, BMPR1A, BRCA1, BRCA2, MEN1, MLH1, MSH2, MSH6, MUTYH, NF2, PMS2, PTEN, RB1, RET, SDHB, SDHC, SDHD, SMAD4, STK11, TP53, TSC1, TSC2, VHL, WT1) that may also be considered for inclusion into the network model¹. Of the 73 genes that the ACMG has identified that have germline variants associated with causative disorders, MT1, PTEN, BRCA1, and RET have been associated with colorectal cancer². For example, The MT1 is a tumor suppressor gene that interacts activated and is activated Akt so that gain-of-function variants of MT1 promote cancer^{3,4}. Over expression WT1 also inhibits cell apoptosis through transcriptional activation of BCL-2 and CCND1 (cyclin D1)⁴. Thus, addition of WT1 will connect gaps in the pathway diagram shown in FIG. 1. RET is a tumor suppression gene that is methylated (suppressed transcription) in 63% of colorectal cancer⁵. Women with BRCA1 mutations have an increased risk of early onset colorectal cancer and has been associated with vincristine drug resistance in colorectal cancer⁶. These pathways may also be incorporated into the model.

Additionally, the network model may be validated based on data from scientific and clinical literature and databases. This includes testing directed at how gene expression (protein level) changes affect the signaling network. A second round of validation of the entire model with retrospective clinical data may also be done using patient sets that are independent of the training data in Phase II.

To increase computational efficiency, one can optimize the solution of the network using stochastic automata network model formalism similar to the methods described in previous studies^{7,8}. This method optimized the matrix-based approach to representing the states and discrete transitions between states described in stochastic automata theory with efficiency improvements that minimize the calculation and use of logical functions.

If one is not satisfied with the model performance during validation, we can apply a ‘normalized graded Boolean model’. Instead of the proteins being simple on or off (0 or 1), one may have fractional amounts to describe the amount of protein. The connects will also be graded with 1 being the basal interaction and interactions increasing or decreasing due to mutations as indicated by the literature and shown in FIG. 1. These interactions will simply be scalar, but if needed can be described using a Hill (or Michaelis Menten) type formalism as done previously by Saucerman et al.⁹. Inhibitory connections and drug action on activity will be performed using a Michaelis-Menten type formalism.

### REFERENCES

1 Kim, J. et al. Prevalence of pathogenic/likely pathogenic variants in the 24 cancer genes of the ACMG Secondary Findings v2.0 list in a large cancer cohort and ethnicity-matched controls. Genome Medicine 10, 99, doi:10.1186/s13073-018-0607-5 (2018).
2 Miller, D. T. et al. ACMG SF v3.0 list for reporting of secondary findings in clinical exome and genome sequencing: a policy statement of the American College of Medical Genetics and Genomics (ACMG). Genetics in medicine: official journal of the American College of Medical Genetics 23, 1381-1390, doi:10.1038/s41436-021-01172-3 (2021).
3 Wang, X. et al. Wilms' tumour suppressor gene 1 (WT1) is involved in the carcinogenesis of Lung cancer through interaction with PI3K/Akt pathway. Cancer cell international 13, 114, doi:10.1186/1475-2867-13-114 (2013).
4 Zhou, B. et al. WT1 facilitates the self-renewal of leukemia-initiating cells through the upregulation of BCL2L2: WT1-BCL2L2 axis as a new acute myeloid leukemia therapy target. Journal of Translational Medicine 18, 254, doi:10.1186/s12967-020-02384-y (2020).
5 Luo, Y. et al. RET is a potential tumor suppressor gene in colorectal cancer. Oncogene 32, 2037-2047, doi:10.1038/onc.2012.225 (2013).
6 Xu, Z. & Zhang, L. BRCA1 expression serves a role in vincristine resistance in colon cancer cells. Oncology letters 14, 345-348, doi:10.3892/ol.2017.6149 (2017).
7 Hoang-Trong, T. M., Ullah, A., Lederer, W. J. & Jafri, M. S. A Stochastic Spatiotemporal Model of Rat Ventricular Myocyte Calcium Dynamics Demonstrated Necessary Features for Calcium Wave Propagation. Membranes 11, 989 (2021).
Jafri, M. S., M. Hoang-Trong, & G. S. Williams. Methods and system for utilizing Markov chain Monte Carlo simulations. U.S. Pat. No. 9,009,095 (2015).
9 Kraeutler, M. J., Soltis, A. R. & Saucerman, J. J. Modeling cardiac β-adrenergic signaling with normalized-Hill differential equations:
comparison with a biochemical model. BMC systems biology 4, 157, doi:10.1186/1752-0509-4-157 (2010).

What is claimed:

1. A method for predicting the response of a cancer patient, based on the presence of one or more variant proteins associated with their cancer, to a chemotherapeutic drug treatment of interest, the method comprising:
(a) variant prediction;
(b) creating a minimum variable product (MVP) that allows a user to evaluate the likely efficacy of a chemotherapy for the cancer patient based on the presence of the one or more variant proteins in the cancer patient; and
(c) developing a prototype that allows the user to select the one or more variant proteins associated with the cancer and a chemotherapeutic drug treatment of interest.

2. The method of claim 1, wherein the variant prediction is used to formulate a Boolean network of a signaling network to predict the outcome of one or more variant profiles and their sensitivity to a chemotherapeutic cancer treatment.

3. The method of claim 1, wherein the creating the MVP allows a user to enter variant information on a patient's cancer and the chemotherapeutic cancer treatment that is to be evaluated for efficacy.

4. The method of claim 1, wherein said variant proteins are those variants known to be associated with a particular cancer.

5. The method of claim 1, wherein said variants are selected from the group consisting of BRAF, KRAS, PTEN, APC, MEK-1, BMPR1A, BRCA1, BRCA2, MEN1, MLH1, MSH2, MSH6, MUTYH, NF2, PMS2, PTEN, RB1, RET, SDHB, SDHC, SDHD, SMAD4, STK11, TP53, TSC1, TSC2, VHL, and WT1.

6. The method of claim 1, wherein the chemotherapeutic drug is selected from the group consisting of an alkylating agent, antimetabolite, anthracycline, topoisomerase inhibitors and mitotic inhibitors.

7. The method of claim 1, wherein the chemotherapeutic drug is selected from the group consisting of vemurafenib, dabrafenib, cetuximab, cobimetinib, trametinib, alpelisib, sorafenib, BAY 1125976 and AZD6482.

8. A system for predicting the response of a cancer patient, based on the presence of one or more variant proteins associated with their cancer, to a chemotherapeutic drug treatment of interest, the system comprising:
at least one processor; and
at least one memory having stored thereon instructions which, when executed by the at least one processor, cause the system at least to perform:
(a) variant prediction;
(b) creating a minimum variable product (MVP) that allows a user to evaluate the likely efficacy of a chemotherapy for the cancer patient based on the presence of the one or more variant proteins in the cancer patient; and
(c) developing a prototype that allows the user to select the one or more variant proteins associated with the cancer and a chemotherapeutic drug treatment of interest.

9. The system of claim 8, wherein the variant prediction is used to formulate a Boolean network of a signaling network to predict the outcome of one or more variant profiles and their sensitivity to a chemotherapeutic cancer treatment.

10. The system of claim 8, wherein the creating the MVP allows a user to enter variant information on a patient's cancer and the chemotherapeutic cancer treatment that is to be evaluated for efficacy.

11. The system of claim 8, wherein said variant proteins are those variants known to be associated with a particular cancer.

12. The system of claim 8, wherein said variants are selected from the group consisting of BRAF, KRAS, PTEN, APC, MEK-1, BMPR1A, BRCA1, BRCA2, MEN1, MLH1, MSH2, MSH6, MUTYH, NF2, PMS2, PTEN, RB1, RET, SDHB, SDHC, SDHD, SMAD4, STK11, TP53, TSC1, TSC2, VHL, and WT1.

13. The system of claim 8, wherein the chemotherapeutic drug is selected from the group consisting of an alkylating agent, antimetabolite, anthracycline, topoisomerase inhibitors and mitotic inhibitor.

14. The system of claim 8, wherein the chemotherapeutic drug is selected from the group consisting of vemurafenib, dabrafenib, cetuximab, cobimetinib, trametinib, alpelisib, sorafenib, BAY 1125976 and AZD6482.

15. A computer-readable medium having stored thereon instructions for predicting the response of a cancer patient, based on the presence of one or more variant proteins associated with their cancer, to a chemotherapeutic drug treatment of interest, the instructions, when executed by at least one processor of a system, cause the system to perform a method comprising:
(a) variant prediction;
(b) creating a minimum variable product (MVP) that allows a user to evaluate the likely efficacy of a chemotherapy for the cancer patient based on the presence of the one or more variant proteins in the cancer patient; and
(c) developing a prototype that allows the user to select the one or more variant proteins associated with the cancer and a chemotherapeutic drug treatment of interest.

16. The computer-readable medium of claim 15, wherein the variant prediction is used to formulate a Boolean network of a signaling network to predict the outcome of one or more variant profiles and their sensitivity to a chemotherapeutic cancer treatment.

17. The computer-readable medium of claim 15, wherein the creating the MVP allows a user to enter variant information on a patient's cancer and the chemotherapeutic cancer treatment that is to be evaluated for efficacy.

18. The computer-readable medium of claim 15, wherein said variant proteins are those variants known to be associated with a particular cancer.

19. The system of claim 15, wherein said variants are selected from the group consisting of BRAF, KRAS, PTEN, APC, MEK-1, BMPR1A, BRCA1, BRCA2, MEN1, MLH1, MSH2, MSH6, MUTYH, NF2, PMS2, PTEN, RB1, RET, SDHB, SDHC, SDHD, SMAD4, STK11, TP53, TSC1, TSC2, VHL, and WT1.

20. The computer-readable medium of claim 15, wherein the chemotherapeutic drug is selected from the group consisting of an alkylating agent, antimetabolite, anthracycline, topoisomerase inhibitors and mitotic inhibitor, and
wherein the chemotherapeutic drug is selected from the group consisting of vemurafenib, dabrafenib, cetuximab, cobimetinib, trametinib, alpelisib, sorafenib, BAY 1125976 and AZD6482.
